(19) United States
(12) Patent Application Publication
(10) Pub. No.: US 2008/0153762 A1
(43) Pub. Date: Jun. 26, 2008

(54) Process for harvesting plants of the apocynaceae family

(75) Inventors: Frederik Michiel Meeuse, Vlaardingen (NL); Kevin John Povey, Purfleet (GB)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco Inc, d/b/a UNILEVER

(21) Appl. No.: 12/002,721

(22) Filed: Dec. 18, 2007

(30) Foreign Application Priority Data
Dec. 21, 2006 (EP) EP06126853
Mar. 16, 2007 (EP) EP07104335

(51) Int. Cl. A61K 31/704; C07J 17/00

(52) U.S. Cl. 514/26; 536/5

(57) ABSTRACT

Process for harvesting plants from the Apocynaceae family comprising the steps of:
(a) removing the plants from the soil,
(b) leaving the intact plants to cure for a period of at least 1 day,
(c) cutting up the cured plants,
(d) further drying the cut plants, to obtain dried plant material comprising the steroidal glycoside having the formula (2):

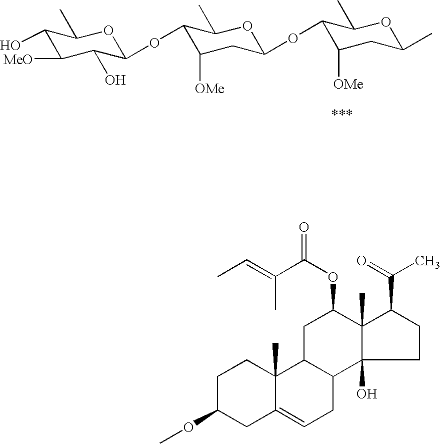

### TECHNICAL FIELD

The present invention relates generally to the field of steroidal glycosides. More in particular, it relates to a process for harvesting plants from the Apocynaceae family, also known as Asclepiadaceae family, which contain steroidal glycosides having appetite suppressant activity and which can be used, for example, in weight management products. The invention especially relates to the harvesting of plants from the Hoodia genus (formerly the Hoodia and Trichocaulon genera).

### BACKGROUND OF THE INVENTION

Extracts obtainable from plants of the Apocynaceae family, also known as Asclepiadaceae family, particularly the Hoodia genus (formerly the Hoodia and Trichocaulon genera) have been shown to have an appetite suppressant activity and are potentially useful in weight management products. U.S. Pat. No. 6,376,657 (CSIR) discloses that these extracts contain steroidal glycosides having the formula 1:

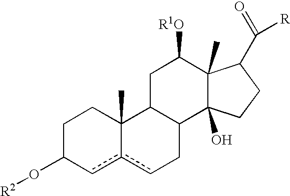

wherein
R=alkyl;
R¹═H, alkyl, tiglyol, benzoyl or any other organic ester group;
R²═H or one or more 6-deoxy carbohydrates, or one or more 2,6-dideoxy carbohydrates, or glucose molecules, or
combinations thereof; and wherein the broken lines indicate the optional presence of a further bond between carbon atoms C4 and C5 or between carbon atoms C5 and C6.

One of the active molecules in one of the purified fractions having good appetite suppressant activity was found to be a compound having the formula 2:

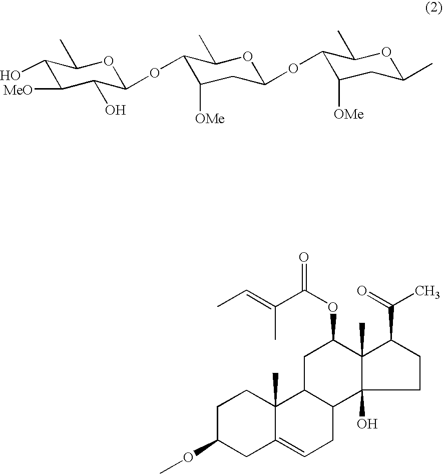

U.S. Pat. No. 6,376,657 also discloses a process to extract the steroidal glycoside having the formula 1 from plants of the Asclepiadaceae family, involving treating plant material with a solvent to extract a fraction having appetite suppressant activity, separating the extraction solution from the rest of the plant material, removing the solvent from the extraction solution and recovering the extract. The patent also discloses methods for synthesizing various steroidal glycosides.

WO2005/116049 (Unilever) discloses that steroidal glycosides can be extracted or separated from undesirable components present in plant material of the Asclepiadaceae (Hoodia) family by means of liquid or supercritical carbon dioxide.

Dried plant material from Hoodia gordonii was milled to a fine powder and subsequently extracted.

US 2005/0202103 discloses Caralluma extracts, wherein the aerial parts of Caralluma plant are dried under shade (on cemented platform).

U.S. Pat. No. 7,008,648 discloses a method of obtaining a plant material from Stapelia and Orbea plants, wherein a suitable method for drying and grinding the original biomass includes either sun drying followed by a heated air-drying or freeze-drying, e.g. lyophilization or chopping of the biomass into small pieces, e.g. 2-10 cm, followed by heated air-drying or freeze-drying.

The purification and isolation of appetite suppressant steroidal glycosides, especially those of Formula 2, from the plants of the Apocynaceae family is costly and cumbersome. It is therefore desirable to improve the yield of steroidal glycosides, especially the steroidal glycosides having the formula 2.

The present inventors have now surprisingly found that the yield of some steroidal glycosides from plants of the Apocynaceae family, in particular the steroidal glycosides having the formula (2), can be improved by the harvesting process according to the present invention, which is characterised in that, after the plants have been removed from the soil, they are left to cure to a moisture content of less than 90% by weight, the cured plants are cut up and further dried, to obtain dried plant material comprising the steroidal glycoside having the formula (2):

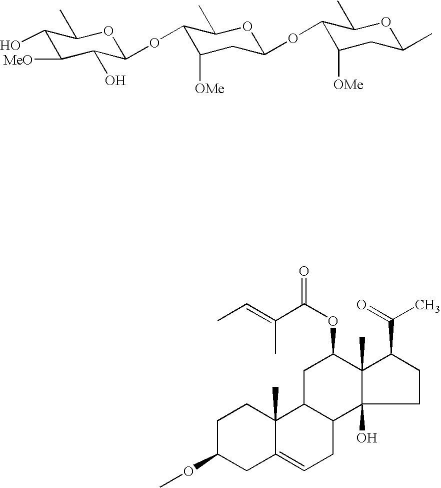

### DEFINITION OF THE INVENTION

The above and other objects are attained by the present invention, which includes, in its first aspect, a process for harvesting plants from the Apocynaceae family comprising the steps of:

(a) removing the plants from the soil,
(b) leaving the intact plants to cure to a moisture content of less than 90% by weight,
(c) cutting up the cured plants,
(d) further drying the cut plants, to obtain dried plant material comprising the steroidal glycoside having the formula (2):

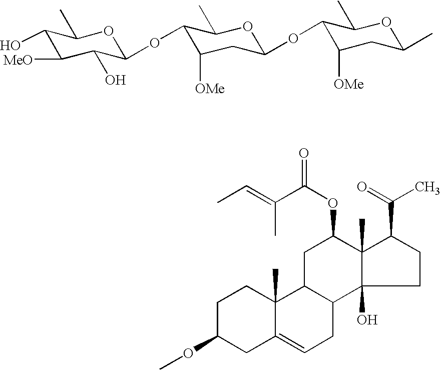

In a second aspect, the invention relates to dried plant material obtainable according to the above process and comprising the steroidal glycoside of Formula 2 in an amount of at least 0.095% by weight.

In a third aspect, the invention relates to extracts and food products comprising the steroidal glycosides having Formula 2.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.”

It should be noted that in specifying any range of concentration or amount, any particular upper concentration can be associated with any particular lower concentration or amount.

For the avoidance of doubt the word “comprising” is intended to mean “including” but not necessarily “consisting of” or “composed of.” In other words, the listed steps or options need not be exhaustive.

“Cut” as used herein means that the size of the plant is reduced, and includes comminuting, pulverizing, etc.

“Mean” as used herein means the average steroidal glycoside content of at least 10 different, randomly selected, plants.

“Curing” as used herein means a process whereby harvested intact plants are simply left until they have achieved a moisture content of less than 90% by weight.

The first aspect of the present invention is a process for harvesting plants of the Apocynaceae family, more preferably the Hoodia family. It is especially preferred if the plant is selected from the group consisting of Trichocaulon piliferum, Trichocaulon officinale, Hoodia currorii, Hoodia gordonii, Hoodia lugardii and mixtures thereof. Hoodia gordonii is especially preferred.

In the first step of the harvesting process, the plants of the Apocynaceae family are completely removed from the soil on which they were grown, preferably including the roots. This can be done either manually by pulling the plants out of the ground (possibly with help of e.g. a spade), or in an automated way, using a suitable harvesting machine or tool.

In the second step, the intact plants are cured, that is, they are simply left until they have achieved a moisture content of less than 90% by weight, preferably less than 88% by weight, more preferably, less than 83% or even less than 80% by weight. The reduction of the moisture level is automatically achieved when the plants are left to cure for a sufficient amount of time. It was found that the curing time should be at least one day, but preferably for at least three days. The maximum time is typically 150 days. A preferred amount of time is from 3 to 100 days, more preferably from 5 days to 50 days, optimally from 7 to 30 days.

Curing is conveniently carried out by simply leaving the intact plants on the soil from which they were removed. No extra measures are needed to obtain the decrease in moisture content and the surprising accompanying relative increase in the amount of the steroidal glycoside having the Formula 2.

Curing can also take place on an area covered with e.g. a shade net, or in a building protected from any climate influences.

After the curing step, the cured plants are subsequently cut. The plants can be cut into any shape, like cubes, slices, julienne, etc. as long as one of the dimensions is less then 30 mm, preferably less then 20 mm, most preferably less then 15 mm. So for a slice or julienne shape the thickness should be less than these dimensions, for a cube all dimensions should be less then these dimensions, etc. Conventional cutting equipment may be used, such as a wood chipper, a bowl cutter or standard food cutting equipment, for example the machines supplied by Urschell, to form cut plant particles. The smaller the size, the faster the subsequent drying time, reducing the possibility of microbial growth. For the cutting step and subsequent drying step, the whole plants may be used, but preferably the plants are used without roots, to minimize the possibility of microbial contamination.

Subsequently, the cut plants are further dried to a much lower moisture content, preferably under conditions whereby direct exposure to UV light is minimized. Suitable drying equipment according to the present invention includes direct and indirect air dryers where the air is heated with any kind of energy source (e.g. electricity, gas, paraffin, energy, etc.). Solar energy heats the air with the sun; the hot air may then be blown into an oven where the material is dried. “Drying” as used herein may include freeze-drying.

Typically, the further drying is conducted at a temperature of from 35 to 120° C., preferably, in order to have optimum drying time, from 50 to 100° C., most preferably from 60 to 100° C. The typical drying period is generally at least 1 hour and may be up to two weeks, preferably from 1 to 72 hours, most preferably from 3 to 48 hours.

In the second drying step, the cut plants are typically dried to a residual moisture content of less than 15% by weight, preferably less than 10% by weight, more preferably less than 5% by weight. The (residual) moisture content can be measured using standard gravimetric techniques or Karl Fischer titration.

The obtained dried plant material, preferably in the form of small pieces or flakes, has a mean total content of steroidal glycosides of at least 1.3% by weight, preferably of at least 1.6% by weight. The dried plant material obtainable according to the invention comprises the steroidal glycoside of Formula 2 in an amount of at least 0.095% by weight (calculated as a mean), preferably at least 0.1%, or even more than 0.15% by weight.

The amount of steroidal glycoside of Formula 2 in the dried plant material can be determined using high performance liquid chromatography (HPLC) with UV detection after extraction or dissolution. Approximately 5 g of material is refluxed with approximately 80 ml of boiling methanol for 1 hour. The resulting extract is filtered and the solid material is washed with methanol. The combined filtrate and washing are transferred to a 100 ml flask and made to volume with methanol. 1 ml of the filtrate is evaporated to dryness and reconstituted in 1 ml acetonitrile/water (50/50 v/v).

Steroidal glycosides are measured by LC-UV at 220 nm. To this end 20 μl of the extracts are injected onto a Zorbax RX-C8 analytical column of 250×4.6 mm packed with 5 μm particles and equipped with a Zorbax RX-C8 guard column of 12.5×4.6 mm packed with the same stationary phase. The column system is held at 40° C. Gradient elution is performed starting at 41.2% acetonitrile/methanol (85/15 v/v) and 58.8% water/methanol (85/15 v/v) at a flow rate of 1 ml/min. Initial conditions are held for 10 minutes before being linearly increased to 88.2% acetonitrile/methanol (85/15 v/v) and 11.8% water/methanol (85/15 v/v) over 30 minutes. After a final hold of 5 minutes, the system is re-equilibrated to the starting conditions.

Compound of Formula 2 of any known purity (95% was used in this case) is used for calibration. Compound 2 may be isolated from an extract of dried Hoodia gordonii using preparative liquid chromatography or may be synthesized (see e.g. U.S. Pat. No. 6,376,657, incorporated by reference herein). A stock solution at 100 μg/ml is prepared in acetonitrile/water (1/1 v/v) and further dilutions are prepared to yield additional calibration standards at 75, 50, 20, 10 and 5 μg/ml. UV response at 220 nm is used for quantification against the Compound 2 calibration line.

In a further embodiment of the process of the present invention, one or more steroidal glycosides are extracted from the dried plant material. Any extraction method may be employed. For instance extraction may be conducted as described in U.S. Pat. No. 6,376,657, incorporated by reference herein. The solvents specifically mentioned to perform the extraction are one or more of methylene chloride (dichloromethane), water, methanol, hexane, ethyl acetate or mixtures thereof. Alternatively, the steroidal glycosides may be extracted using liquid or supercritical carbon dioxide such as described in WO2005/116049 (Unilever).

The dried plant material or the extract therefrom can be used in appetite suppressant food products, and this constitutes a third aspect of the present invention. Examples of such food products are beverages, snacks, bars, spreads, dressings, soups, etc., or meal replacement products, which can be used in the management of body weight or in the dietary control of obesity.

All amounts, parts, ratios and percentages used herein are by weight, unless otherwise specified.

While the above summarizes the present invention, it will become apparent to those skilled in the art that modifications, variations and alterations may be made without deviating from the scope and spirit of the present invention as described and claimed herein. The invention will now be further illustrated in the following, non-limiting example.

### EXAMPLE 1

28 small Hoodia gordonii plants having an average weight of 0.5 kg were harvested. 138 medium Hoodia gordonii plants having an average weight of 1.4 kg were harvested. They were randomly divided over 2 groups, group S containing 50 plants and group O containing 78 plants. 10 small, large and medium plants from group O were cut into small pieces and oven dried. 10 medium plants from group S were cut into small pieces and sun dried.

All other plants were first “sun cured” for a different number of days, up to 60 days. After the curing the % fresh weight of all plants was determined. Then the small, large and medium plants from group O were cut into small pieces and oven dried. The medium plants from group S were cut into small pieces and sun dried.

Of all plants the level of compound (2) was determined. Table 1 shows of all plant the initial weight, the weight after curing and the compound (2) level. Surprisingly we found that there is a correlation between the fraction of the weight loss due to curing (defined as “Fraction” below) and the active level, indicating the beneficial effect of curing on the compound (2) level.

Fraction=(Initial Weight−Final Weight)/Initial Weight

Final weight herein is the weight after curing. The correlation is given by the following equation:

compound(2)=exp(A+B*(1−Fraction))

The initial moisture content is typically 90%. In such case, the above correlation (wherein Fraction is re-worked to % moisture after curing) can then be modified to:

compound(2)=exp(A+B*(10/(100−% moisture))

Table 2 shows the values for A and B, including their standard error and the P-value for the correlations for the small, medium group O, medium group S and large plants.

TABLE 1

| | | Final | | |
|---|---|---|---|---|
| | Initial Weight | Weight | | |
| | (kg) | (kg) | Fraction | compound (2) |
| Large | 3.93 | 3.93 | 0.000 | 0.078 |
| Large | 6.01 | 6.01 | 0.000 | 0.098 |
| Large | 6.40 | 6.40 | 0.000 | 0.070 |
| Large | 4.55 | 4.55 | 0.000 | 0.089 |
| Large | 2.02 | 2.02 | 0.000 | 0.092 |
| Large | 3.50 | 3.50 | 0.000 | 0.062 |
| Large | 3.66 | 3.66 | 0.000 | 0.065 |
| Large | 4.35 | 4.35 | 0.000 | 0.153 |
| Large | 6.47 | 6.47 | 0.000 | 0.052 |
| Large | 3.85 | 3.85 | 0.000 | 0.054 |
| Large | 4.96 | 2.52 | 0.492 | 0.124 |
| Large | 2.63 | 0.48 | 0.819 | 0.100 |
| Large | 4.53 | 2.35 | 0.481 | 0.156 |
| Large | 5.97 | 0.47 | 0.921 | 0.121 |
| Large | 7.94 | 0.39 | 0.951 | 0.136 |
| Large | 4.73 | 2.34 | 0.505 | 0.146 |
| Large | 4.98 | 2.49 | 0.500 | 0.095 |
| Large | 3.35 | 0.46 | 0.864 | 0.165 |
| Large | 6.98 | 3.35 | 0.520 | 0.133 |
| Large | 4.67 | 2.22 | 0.525 | 0.147 |
| Large | 5.32 | 0.22 | 0.959 | 0.279 |
| Large | 3.11 | 0.16 | 0.948 | 0.193 |
| Large | 5.73 | 0.16 | 0.972 | 0.177 |
| Large | 4.85 | 0.21 | 0.957 | 0.228 |
| Large | 2.90 | 0.16 | 0.944 | 0.161 |
| Large | 3.78 | 0.87 | 0.770 | 0.221 |
| Large | 3.97 | 1.06 | 0.733 | 0.089 |
| Large | 3.90 | 0.90 | 0.769 | 0.134 |
| Large | 4.67 | 1.25 | 0.732 | 0.129 |
| Large | 4.55 | 0.20 | 0.956 | 0.115 |
| Medium, group O | 2.89 | 2.89 | 0.000 | 0.156 |
| Medium, group O | 2.37 | 2.37 | 0.000 | 0.248 |
| Medium, group O | 1.79 | 1.79 | 0.000 | 0.107 |
| Medium, group O | 1.65 | 1.65 | 0.000 | 0.071 |
| Medium, group O | 1.77 | 1.77 | 0.000 | 0.066 |
| Medium, group O | 1.62 | 1.62 | 0.000 | 0.061 |
| Medium, group O | 1.66 | 1.66 | 0.000 | 0.062 |
| Medium, group O | 1.80 | 1.80 | 0.000 | 0.114 |
| Medium, group O | 0.69 | 0.69 | 0.000 | 0.182 |
| Medium, group O | 1.57 | 1.57 | 0.000 | 0.096 |
| Medium, group O | 1.70 | 1.31 | 0.229 | 0.079 |
| Medium, group O | 1.29 | 0.92 | 0.285 | 0.037 |
| Medium, group O | 1.38 | 1.06 | 0.231 | 0.043 |
| Medium, group O | 0.78 | 0.60 | 0.227 | 0.048 |
| Medium, group O | 1.42 | 1.09 | 0.232 | 0.098 |
| Medium, group O | 0.93 | 0.72 | 0.226 | 0.071 |
| Medium, group O | 1.04 | 0.78 | 0.249 | 0.098 |
| Medium, group O | 0.86 | 0.66 | 0.231 | 0.076 |
| Medium, group O | 1.59 | 1.21 | 0.239 | 0.115 |
| Medium, group O | 1.49 | 1.15 | 0.227 | 0.096 |
| Medium, group O | 1.24 | 0.74 | 0.402 | 0.119 |
| Medium, group O | 1.56 | 0.64 | 0.590 | 0.143 |
| Medium, group O | 1.79 | 0.70 | 0.610 | 0.068 |
| Medium, group O | 1.03 | 0.54 | 0.472 | 0.092 |
| Medium, group O | 1.02 | 0.61 | 0.406 | 0.050 |
| Medium, group O | 1.23 | 0.64 | 0.481 | 0.119 |
| Medium, group O | 1.02 | 0.50 | 0.507 | 0.192 |
| Medium, group O | 0.95 | 0.40 | 0.579 | 0.140 |
| Medium, group O | 0.89 | 0.46 | 0.483 | 0.082 |
| Medium, group O | 1.74 | 0.90 | 0.483 | 0.128 |
| Medium, group O | 2.09 | 0.88 | 0.579 | 0.105 |
| Medium, group O | 2.65 | 1.05 | 0.603 | 0.198 |
| Medium, group O | 1.95 | 0.88 | 0.549 | 0.128 |
| Medium, group O | 1.34 | 0.54 | 0.596 | 0.184 |
| Medium, group O | 1.50 | 0.24 | 0.840 | 0.099 |
| Medium, group O | 1.89 | 0.78 | 0.587 | 0.169 |
| Medium, group O | 1.52 | 0.58 | 0.618 | 0.162 |
| Medium, group O | 2.07 | 1.00 | 0.516 | 0.138 |
| Medium, group O | 1.27 | 0.64 | 0.494 | 0.196 |
| Medium, group O | 1.49 | 0.86 | 0.423 | 0.173 |
| Medium, group O | 1.35 | 0.47 | 0.651 | 0.232 |
| Medium, group O | 1.53 | 0.31 | 0.798 | 0.128 |
| Medium, group O | 1.61 | 0.86 | 0.467 | 0.113 |
| Medium, group O | 1.47 | 0.55 | 0.625 | 0.097 |
| Medium, group O | 1.64 | 0.77 | 0.531 | 0.322 |
| Medium, group O | 0.92 | 0.25 | 0.727 | 0.145 |
| Medium, group O | 1.06 | 0.42 | 0.605 | 0.188 |
| Medium, group O | 1.23 | 0.11 | 0.914 | 0.116 |
| Medium, group O | 1.49 | 0.56 | 0.624 | 0.302 |
| Medium, group O | 1.72 | 1.03 | 0.403 | 0.232 |
| Medium, group O | 1.53 | 1.18 | 0.229 | 0.059 |
| Medium, group O | 1.21 | 0.93 | 0.233 | 0.118 |
| Medium, group O | 1.37 | 1.04 | 0.243 | 0.080 |
| Medium, group O | 1.17 | 0.90 | 0.229 | 0.106 |
| Medium, group O | 1.28 | 0.99 | 0.228 | 0.106 |
| Medium, group O | 1.32 | 1.01 | 0.234 | 0.127 |
| Medium, group O | 1.14 | 0.87 | 0.234 | 0.075 |
| Medium, group O | 1.80 | 1.37 | 0.239 | 0.132 |
| Medium, group O | 1.69 | 1.25 | 0.258 | 0.132 |
| Medium, group O | 0.94 | 0.71 | 0.245 | 0.160 |
| Medium, group O | 1.11 | 0.42 | 0.625 | 0.130 |
| Medium, group O | 1.03 | 0.39 | 0.622 | 0.046 |
| Medium, group O | 1.38 | 0.25 | 0.821 | 0.196 |
| Medium, group O | 1.59 | 0.63 | 0.604 | 0.147 |
| Medium, group O | 1.67 | 0.37 | 0.777 | 0.056 |
| Medium, group O | 1.50 | 0.28 | 0.814 | 0.221 |
| Medium, group O | 1.52 | 0.37 | 0.758 | 0.239 |
| Medium, group O | 1.24 | 0.36 | 0.713 | 0.059 |
| Medium, group O | 1.35 | 0.56 | 0.585 | 0.100 |
| Medium, group O | 0.88 | 0.31 | 0.646 | 0.109 |
| Medium, group O | 1.74 | 0.43 | 0.752 | 0.137 |
| Medium, group O | 1.63 | 0.22 | 0.864 | 0.118 |
| Medium, group O | 1.35 | 0.35 | 0.742 | 0.073 |
| Medium, group O | 1.68 | 0.20 | 0.883 | 0.064 |
| Medium, group O | 0.98 | 0.20 | 0.795 | 0.070 |
| Medium, group O | 1.79 | 0.21 | 0.881 | 0.286 |
| Medium, group O | 1.30 | 0.08 | 0.935 | 0.152 |
| Medium, group O | 1.40 | 0.37 | 0.735 | 0.175 |
| Medium, group O | 1.15 | 0.29 | 0.747 | 0.097 |
| Medium, group O | 2.05 | 0.14 | 0.933 | 0.074 |
| Medium, group O | 0.50 | 0.50 | 0.000 | 0.135 |
| Medium, group O | 0.40 | 0.40 | 0.000 | 0.036 |
| Medium, group O | 0.26 | 0.26 | 0.000 | 0.039 |
| Medium, group O | 0.29 | 0.29 | 0.000 | 0.044 |
| Medium, group O | 0.49 | 0.49 | 0.000 | 0.052 |
| Medium, group O | 0.60 | 0.60 | 0.000 | 0.082 |
| Medium, group O | 0.64 | 0.64 | 0.000 | 0.146 |
| Medium, group O | 0.16 | 0.16 | 0.000 | 0.048 |
| Medium, group O | 0.56 | 0.56 | 0.000 | 0.070 |
| Medium, group O | 0.48 | 0.48 | 0.000 | 0.059 |
| Medium, group O | 0.72 | 0.34 | 0.530 | 0.256 |
| Medium, group O | 0.19 | 0.10 | 0.474 | 0.103 |
| Medium, group O | 0.66 | 0.30 | 0.544 | 0.113 |
| Medium, group O | 0.55 | 0.12 | 0.781 | 0.078 |
| Medium, group O | 0.40 | 0.18 | 0.552 | 0.069 |
| Medium, group O | 0.44 | 0.22 | 0.500 | 0.039 |
| Medium, group O | 0.64 | 0.33 | 0.488 | 0.112 |
| Medium, group O | 0.42 | 0.21 | 0.502 | 0.125 |
| Medium, group O | 0.32 | 0.17 | 0.465 | 0.097 |
| Medium, group O | 0.21 | 0.10 | 0.515 | 0.243 |
| Medium, group O | 0.72 | 0.10 | 0.860 | 0.090 |
| Medium, group O | 0.72 | 0.24 | 0.666 | 0.070 |
| Medium, group O | 0.31 | 0.08 | 0.739 | 0.156 |
| Medium, group O | 0.58 | 0.11 | 0.810 | 0.123 |
| Medium, group O | 1.01 | 0.26 | 0.743 | 0.096 |
| Medium, group O | 0.71 | 0.14 | 0.804 | 0.091 |
| Medium, group O | 0.34 | 0.09 | 0.734 | 0.185 |
| Medium, group O | 0.18 | 0.05 | 0.725 | 0.101 |
| Medium, group S | 1.52 | 1.52 | 0.000 | 0.050 |
| Medium, group S | 1.29 | 1.29 | 0.000 | 0.037 |
| Medium, group S | 0.85 | 0.85 | 0.000 | 0.058 |
| Medium, group S | 1.25 | 1.25 | 0.000 | 0.066 |
| Medium, group S | 1.11 | 1.11 | 0.000 | 0.034 |
| Medium, group S | 0.87 | 0.87 | 0.000 | 0.034 |
| Medium, group S | 1.63 | 1.63 | 0.000 | 0.028 |
| Medium, group S | 0.91 | 0.91 | 0.000 | 0.051 |
| Medium, group S | 1.26 | 1.26 | 0.000 | 0.041 |
| Medium, group S | 2.16 | 2.16 | 0.000 | 0.077 |
| Medium, group S | 1.27 | 0.95 | 0.251 | 0.038 |
| Medium, group S | 1.36 | 1.03 | 0.245 | 0.171 |
| Medium, group S | 2.20 | 1.70 | 0.228 | 0.135 |
| Medium, group S | 1.48 | 1.15 | 0.225 | 0.121 |
| Medium, group S | 1.91 | 1.39 | 0.274 | 0.082 |
| Medium, group S | 1.41 | 1.04 | 0.263 | 0.067 |
| Medium, group S | 1.43 | 1.10 | 0.231 | 0.088 |
| Medium, group S | 1.29 | 1.00 | 0.230 | 0.037 |
| Medium, group S | 1.31 | 0.98 | 0.250 | 0.062 |
| Medium, group S | 0.96 | 0.73 | 0.237 | 0.234 |
| Medium, group S | 1.70 | 0.89 | 0.477 | 0.038 |
| Medium, group S | 1.08 | 0.40 | 0.629 | 0.171 |
| Medium, group S | 1.05 | 0.54 | 0.485 | 0.135 |
| Medium, group S | 1.23 | 0.58 | 0.528 | 0.121 |
| Medium, group S | 1.60 | 0.45 | 0.717 | 0.082 |
| Medium, group S | 1.14 | 0.46 | 0.598 | 0.067 |
| Medium, group S | 0.95 | 0.45 | 0.524 | 0.088 |
| Medium, group S | 1.39 | 0.71 | 0.490 | 0.037 |
| Medium, group S | 1.08 | 0.53 | 0.511 | 0.062 |
| Medium, group S | 0.90 | 0.65 | 0.278 | 0.234 |
| Medium, group S | 1.29 | 0.77 | 0.405 | 0.241 |
| Medium, group S | 1.03 | 0.43 | 0.583 | 0.160 |
| Medium, group S | 0.83 | 0.35 | 0.576 | 0.076 |
| Medium, group S | 1.20 | 0.32 | 0.731 | 0.290 |
| Medium, group S | 1.36 | 0.51 | 0.626 | 0.115 |
| Medium, group S | 1.58 | 0.94 | 0.407 | 0.218 |
| Medium, group S | 2.39 | 0.97 | 0.594 | 0.165 |
| Medium, group S | 0.88 | 0.46 | 0.480 | 0.142 |
| Medium, group S | 0.95 | 0.39 | 0.589 | 0.094 |
| Medium, group S | 1.81 | 0.83 | 0.541 | 0.156 |
| Medium, group S | 1.40 | 0.68 | 0.515 | 0.126 |
| Medium, group S | 0.83 | 0.23 | 0.724 | 0.147 |
| Medium, group S | 1.92 | 0.16 | 0.917 | 0.196 |
| Medium, group S | 1.10 | 0.46 | 0.583 | 0.279 |
| Medium, group S | 1.55 | 0.14 | 0.910 | 0.239 |
| Medium, group S | 0.86 | 0.16 | 0.814 | 0.310 |
| Medium, group S | 2.56 | 0.22 | 0.915 | 0.132 |
| Medium, group S | 1.71 | 0.40 | 0.766 | 0.159 |
| Medium, group S | 2.01 | 0.12 | 0.940 | 0.087 |
| Medium, group S | 1.03 | 0.27 | 0.739 | 0.069 |
| Small | 0.50 | 0.50 | 0.000 | 0.135 |
| Small | 0.40 | 0.40 | 0.000 | 0.036 |
| Small | 0.26 | 0.26 | 0.000 | 0.039 |
| Small | 0.29 | 0.29 | 0.000 | 0.044 |
| Small | 0.49 | 0.49 | 0.000 | 0.052 |
| Small | 0.60 | 0.60 | 0.000 | 0.082 |
| Small | 0.64 | 0.64 | 0.000 | 0.146 |
| Small | 0.16 | 0.16 | 0.000 | 0.048 |
| Small | 0.56 | 0.56 | 0.000 | 0.070 |
| Small | 0.48 | 0.48 | 0.000 | 0.059 |
| Small | 0.72 | 0.34 | 0.530 | 0.256 |
| Small | 0.19 | 0.10 | 0.474 | 0.103 |
| Small | 0.66 | 0.30 | 0.544 | 0.113 |
| Small | 0.55 | 0.12 | 0.781 | 0.078 |
| Small | 0.40 | 0.18 | 0.552 | 0.069 |
| Small | 0.44 | 0.22 | 0.500 | 0.039 |
| Small | 0.64 | 0.33 | 0.488 | 0.112 |
| Small | 0.42 | 0.21 | 0.502 | 0.125 |
| Small | 0.32 | 0.17 | 0.465 | 0.097 |
| Small | 0.21 | 0.10 | 0.515 | 0.243 |
| Small | 0.72 | 0.10 | 0.860 | 0.090 |
| Small | 0.72 | 0.24 | 0.666 | 0.070 |
| Small | 0.31 | 0.08 | 0.739 | 0.156 |
| Small | 0.58 | 0.11 | 0.810 | 0.123 |
| Small | 1.01 | 0.26 | 0.743 | 0.096 |
| Small | 0.71 | 0.14 | 0.804 | 0.091 |
| Small | 0.34 | 0.09 | 0.734 | 0.185 |
| Small | 0.18 | 0.05 | 0.725 | 0.101 |

TABLE 2

| | A | | | B | | |
|---|---|---|---|---|---|---|
| | estimate | error | P | estimate | error | P |
| Small | −2.7160 | 0.1457 | <0.0001 | 0.7331 | 0.2800 | 0.0146 |
| Medium, O | −2.3751 | 0.1609 | 0.0015 | 0.4749 | 0.3198 | 0.0015 |
| Medium, S | −2.8983 | 0.1214 | <0.0001 | 1.5095 | 0.2448 | <0.0001 |
| Large | −2.5588 | 0.0864 | <0.0001 | 0.9672 | 0.1578 | <0.0001 |

1. Process for harvesting plants from the Apocynaceae family comprising the steps of:
(a) removing the plants from the soil,
(b) leaving the intact plants to cure to a moisture content of less than 90% by weight,
(c) cutting up the cured plants,
(d) further drying the cut plants, to obtain dried plant material comprising the steroidal glycoside having the formula (2):

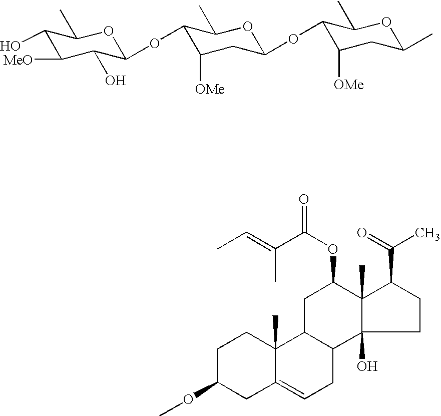

2. Process according to claim 1, whereby the plants are left to cure for a period of at least about 1 day.

3. Process according to claim 1 whereby the plants are left to cure for a period of about 1 to about 150 days.

4. Process according to claim 1 whereby the plants are left to cure for a period of about 3 to about 100 days.

5. Process according to claim 1, whereby the plants are left to cure in the open air for a period of about 7 to about 30 days.

6. Process according to claim 1, wherein the cured plants are subsequently dried to a residual moisture content of less than about 15% by weight.

7. Process according to claim 1, wherein the subsequent drying is conducted at a temperature of from about 35° C. to about 120° C.

8. Process according to claim 1, wherein the plants are selected from the group consisting of Hoodia gordonii, Hoodia currorii, Hoodia lugardii and mixtures thereof.

9. Process according to claim 8, wherein the plant is Hoodia gordonii.

10. Process according to claim 1, wherein the dried plant material further comprises one or more other steroidal glycosides having the formula: 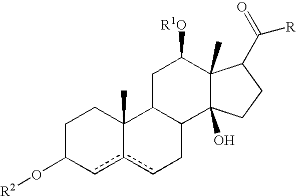
wherein R=alkyl;
R1=H, alkyl, tiglyol, benzoyl or any other organic ester group;
R2=H or one or more 6-deoxy carbohydrates, or one or more 2,6-dideoxy carbohydrates, or glucose molecules, or combinations thereof; and wherein the broken lines indicate the optional presence of a further bond between carbon atoms C4 and C5 or between carbon atoms C5 and C6, or mixtures thereof.

11. Process according to claim 1 wherein the dried plant material has a mean total steroidal glycoside content of at least about 1.3% by weight, based on anhydrous dried plant material.

12. Process according to claim 1 wherein the individual dried plant material has a total steroidal glycoside content of at least about 1.6% by weight, based on anhydrous dried plant material.

13. Process according to claim 1 wherein the dried plant material comprises at least about 0.095% by weight of the steroidal glycoside of Formula 2, based on anhydrous dried plant material.

14. Dried plant material obtainable according to the process of claim 1 comprising at least about 0.095% by weight of the steroidal glycoside of Formula 2, based on anhydrous dried plant material.

15. Food product comprising the dried plant material obtainable according to the process of claim 1.

16. Process according to claim 1, further comprising obtaining an extract from the dried plant material.

17. An extract obtainable according to the process of claim 16.

18. Food product comprising an extract of claim 17.

19. Food product according to claim 18, in the form of a beverage, snack, bar, spread, dressing, soup or meal replacement product.

20. A method according to claim 1, wherein the steroidal glycosides are to be used in the management of body weight or in the dietary control of obesity.
